(19) United States
(12) Patent Application Publication
(10) Pub. No.: US 2025/0251520 A1
(43) Pub. Date: Aug. 7, 2025

(54) SIGNAL PROCESSING ARRANGEMENT, PHOTON COUNTING CIRCUITRY, DEVICE FOR MEDICAL DIAGNOSTICS AND SIGNAL PROCESSING METHOD

(71) Applicant: ams International AG, Jona (CH)

(72) Inventors: Srinidhi Koushik KANAGAL RAMESH, PFÄFFIKON (CH); Roger STEADMAN BOOKER, Aachen (DE); Fridolin MICHEL, Au (CH)

(21) Appl. No.: 18/856,627

(22) PCT Filed: Mar. 23, 2023
(86) PCT No.: PCT/EP2023/057479

§ 371 Date: Oct. 14, 2024

(30) Foreign Application Priority Data
Apr. 20, 2022 (DE) 102022109535.5

(51) Int. Cl. G01T 1/17; A61B 6/42

(52) U.S. Cl. CPC G01T 1/17; A61B 6/4241

(57) ABSTRACT

A signal processing arrangement for an electromagnetic radiation sensor application includes a common input, auto-zero, AZ, comparators. Each AZ comparator comprises a signal input for receiving a voltage to be compared and a signal output coupled to a processing block. The processing block evaluates comparison results from the AZ comparators. An AZ DAC provides an AZ reference voltage at an AZ reference output. Switching circuitry individually couples the common input and the AZ reference output to the signal input of each of the AZ comparators in a switchable fashion. A control block disconnects, by controlling the switching circuitry, for each of the AZ comparators in a non-overlapping fashion, the common input from the signal input of the respective AZ comparator during a respective disconnection time, and connect the AZ reference output within the respective disconnection time to the signal input of the respective AZ comparator for a respective AZ time.

### RELATED APPLICATIONS

This application is a National Stage application, filed under 35 U.S.C. § 371, of International Patent Application No. PCT/EP2023/057479, filed on Mar. 23, 2023, and claims priority under 35 U.S.C. § 119 (a) and 35 U.S.C. § 365 (b) from German Patent Application No. 10 2022 109 535.5, filed Apr. 20, 2022; the above applications are incorporated by reference herein in their entirety.

### TECHNICAL FIELD

The disclosure relates to a signal processing arrangement for an electromagnetic radiation sensor application, such as a multi-energy spectral CT (computed tomography). The disclosure further relates to a photon counting circuitry, a device for medical diagnostics and to a signal processing method.

### BACKGROUND

Electromagnetic radiation sensor applications, such as multi-energy spectral CT, regularly use photon counting systems for every pixel, respectively channel, of the image to be produced. Every pixel, respectively channel, includes several comparators for evaluating the pulse-like signals provided by a frontend circuit evaluating the signal of a radiation-sensitive device like a photodiode. Due to very tight offset and/or gain error specifications, the offset of each comparator, optionally along with a gain error of the frontend, needs to be removed. In conventional approaches this is accomplished by means of a digital-to-analog converter, DAC, which is local to every comparator. Each of these DACs occupies a significant chip area and consumes power.

### SUMMARY

Various embodiments of the present disclosure relate to an improved processing concept that allows a more efficient processing of input signals, in particular in electromagnetic radiation sensor applications.

In one or more examples, the improved processing concept is based on the idea that instead of providing a separate DAC for each comparator, only a single DAC for a plurality of comparators is used. To this end, switching circuitry and a control logic are provided that individually provide the output voltage of the single DAC to each of the comparators within a time period during which the signal input of the respective comparator is disconnected from a common input, to which for example a pulse-shaped signal is provided from the frontend.

For example, the plurality of comparators are auto-zero, AZ, comparators such that the voltage provided by the DAC is an AZ voltage generated based on a predetermined AZ word that can be individual for each of the AZ comparators.

With the provision of a single DAC only, chip area and power consumption can be reduced compared to conventional approaches.

In an example implementation of a signal processing arrangement for an electromagnetic radiation sensor application according to the improved processing concept, the signal processing arrangement comprises a common input that, for example, can be coupled to the output of a frontend circuit of the electromagnetic radiation sensor application providing a signal with voltage pulses. The signal processing arrangement further comprises a plurality of AZ comparators, each comprising a reference potential terminal for receiving a reference voltage, a signal input for receiving a voltage to be compared, and a signal output coupled to a processing block.

The signal processing arrangement further comprises the processing block that is configured to evaluate comparison results provided by the plurality of AZ comparators, an AZ DAC being configured to provide an AZ reference voltage at an AZ reference output based on a predetermined AZ word, and switching circuitry that individually couples the common input and the AZ reference output to the signal input of each of the AZ comparators in a switchable fashion. This allows that, for each AZ comparator, the signal input can be connected to the common input and/or to the AZ reference output independent of the connection state of the other AZ comparators.

The signal processing arrangement further comprises a control block that is configured to disconnect, by controlling the switching circuitry, for each of the AZ comparators, e.g. in a non-overlapping fashion, the common input from the signal input of the respective AZ comparator during a respective disconnection time, and to connect, by controlling the switching circuitry, the AZ reference output within the respective disconnection time to the signal input of the respective AZ comparator for a respective AZ time.

This can trigger an AZ operation in the respective AZ comparator. The AZ operation might in itself require couple of hundreds of ns to complete and as such might need to be refreshed every few us in order not to compromise the drift performance. However in this case the radiation dosage, e.g. X-ray dosage, during the AZ time is effectively wasted and as such can lead to a significant dead time which is undesirable.

Hence, if the signal processing arrangement comprises N+1 AZ comparators, at each time at least N AZ comparators are connected to the common input. This also means that these at least N AZ comparators are operable for providing a comparison result while only one or even no AZ comparator is inoperable at a time due to the disconnection of the common input. This aspect allows for reducing the dead time significantly.

During the respective AZ time, which lies within the respective disconnection time, the AZ reference voltage for the respective comparator is provided to its signal input, thereby allowing an AZ operation, which removes offset and/or low frequency noise.

For example, the control block is configured to perform the disconnecting of each of the AZ comparators sequentially. This allows a ping-pong scheme to be applied to auto-zero the offset and/or low frequency noise of one of the comparators, while the rest are operational. By doing so there is little dead time and a good low frequency noise/drift performance can still be achieved. Sequential disconnecting may include that two or more AZ comparators are disconnected at a time. This may be under the condition that a sufficient number of AZ comparators remains operable for providing a comparison result.

For example, the sequential disconnecting is performed repeatedly with a predetermined repetition time. The shorter the repetition time, the more often one of the comparators is non-operational at a time. If the repetition time is too long, the effects of the auto-zeroing may be reduced. Hence, the repetition time may be chosen to consider both of the two mentioned conditions.

In various implementations, the processing block is configured to count events based on the comparison results provided by the plurality of AZ comparators. This can be particularly useful in photon counting applications.

For example, a number of the AZ comparators in the range from 4 to 10, e.g. in the range from 5 to 8.

During operation of the signal processing arrangement at the end of a respective disconnection time, e.g., when the signal input of the AZ comparator is reconnected to the common input, a voltage jump or voltage step may occur depending on the voltage level at the common input. In some cases this may generate a glitch at the signal input of the reconnected AZ comparator, or even at the common input, respectively the signal input of all comparators. In a large multi-pixel CT system there could be thousands of comparators switching per IC which makes it important to reduce glitch and/or crosstalk from supplies and/or reference paths.

If such glitches have to be considered, the improved processing concept provides approaches on how to handle those glitches.

For example, operation of the processing block, respectively evaluating the comparison results, may be halted at an end of the respective disconnection times. Hence, any comparison results resulting from potential glitches are not taken into account by the processing block.

For example, to this end the control block is further configured to provide a halt indicator to the processing block at an end of the respective disconnection time, and the processing block is configured to halt evaluating of the comparison results during provision of the halt indicator.

Another example of a method to handle glitches according to the improved processing concept is to provide the voltage at the common input to the signal input of the AZ comparator via a decoupling element after the AZ comparator is auto-zeroed, e.g., after the respective AZ time, but still within the disconnection time. This achieves that the same or at least a similar voltage as at the common input is present at the signal input of the disconnected AZ comparator when reconnecting at the end of the disconnection time. The decoupling element, for example, is a buffer element. Due to the same or similar voltage at the time of the reconnection, glitches can be avoided or at least reduced.

For example, to this end the switching circuitry further individually couples the common input to the signal input of each of the AZ comparators in a switchable fashion via a decoupling element. Furthermore, the control block is configured to connect, by controlling the switching circuitry, the common input via the decoupling element to the signal input of the respective AZ comparator within the respective disconnection time and after the respective AZ time. In particular, both conditions are to be met.

Both the example implementations of handling glitches, e.g., halting operation of the processing block and connecting the common input via a decoupling element to the signal input, can be combined.

As mentioned above, the halt indicator is provided at the end of the respective disconnection time. This means that the halt indicator, for example, is provided from briefly before the end of the disconnection time to briefly after the end of the disconnection time, or in other words, the end of the disconnection time lies within the duration of the halt indicator.

For example, if only the approach with the halt indicator is used, e.g., without employing a decoupling element, a duration of the halt indicator may be in the range between 500 ps and 5000 ps, for example in the range between 1 ns and 2 ns.

If both approaches for handling glitches are combined, e.g., with employing the decoupling element, a duration of the halt indicator may be in the range between 50 ps and 500 ps, for example in the range between 100 ps and 200 ps.

Each AZ comparator, for example, comprises an AZ capacitor connected between a reference input of the respective AZ comparator and the respective reference potential terminal.

The AZ comparator further comprises a switchable feedback connection between the respective reference input and the respective signal output, wherein the control block is configured to enable the respective feedback connection during the respective AZ time.

Hence, during operation of the AZ comparator, during the respective AZ time, when the AZ voltage is provided to the signal input and the connection between the reference input and the signal output is established, the AZ reference voltage provided at the signal input will establish at the reference input and charge the AZ capacitor to this AZ reference voltage. Hence, after opening the connection between the reference input and the signal output of the comparator, the AZ capacitor determines the voltage at the reference input and has influence on the threshold voltage of the comparator.

For example, the AZ word is different for each AZ comparator. Hence, the respective AZ reference voltage will also be different for each AZ comparator, allowing to individually tune and/or compensate the threshold voltage of each comparator.

A photon counting circuitry according to the improved processing concept comprises a signal processing arrangement according to one of the embodiments described above. The photon counting circuitry further comprises a photon detector being configured to generate a current pulse upon reception of a photon and a signal shaper coupled to the photon detector and configured to generate a voltage pulse at the common input based on a received current pulse. For example, the photon detector has a photon-sensitive area and is configured to generate a current pulse, when a photon hits the photon-sensitive area.

A device for medical diagnostics according to the improved processing concept comprises at least one photon counting circuitry as described above. The device is, for example, configured as an X-ray apparatus or a computed tomography scanner.

The improved processing concept is not limited to the apparatus-based implementations but can also be implemented with a corresponding signal processing method.

For example, a signal processing method according to the improved processing concept is provided for an electromagnetic radiation sensor application with a common input and a plurality of AZ comparators, wherein each of the AZ comparators comprises a signal input for receiving a voltage to be compared, the signal input being switchably coupled to the common input, and a signal output for providing a comparison result. The method comprises disconnecting, for each of the AZ comparators, e.g. in a non-overlapping fashion, the common input from the signal input of the respective AZ comparator during a respective disconnection time, providing, within the respective disconnection time, an AZ reference voltage based on a respective predetermined AZ word to the signal input of the respective AZ comparator for a respective AZ time, and evaluating of the comparison results provided by the plurality of AZ comparators.

Referring to the various implementations of the signal processing arrangement described above, the effect of glitching may occur at the end, respectively after, the respective disconnection time, if the signal input of a comparator is reconnected to the common input. In order to consider such glitching effects, the method may further comprise providing a halt indicator at an end of the respective disconnection time and halting evaluating of the comparison results during provision of the halt indicator.

In addition or as an alternative, the method may further comprise connecting the common input via a decoupling element to the signal input of the respective AZ comparator within the respective disconnection time and after the respective AZ time.

Further implementations of the signal processing method become readily apparent for the skilled reader from the description of the various implementations of the signal processing arrangement above. For example, the method may be implemented in a signal processing arrangement as described above. Specifically, the method may be implemented in a photon counting system where there are one or more comparators additionally deployed than the minimum number of comparators necessary for the application. Hence there is always, e.g., at any time during operation, a minimum number of comparators that are operational for comparison. However, further implementations are not excluded by these examples.

### BRIEF DESCRIPTION OF DRAWINGS

Embodiments related to the improved processing concept will be explained in more detail in the following with the aid of the drawings. Elements and functional blocks having the same or similar function bear the same reference numerals throughout the drawings. Hence their description is not necessarily repeated in the following drawings.

In the drawings:

FIG. 1 shows an example implementation of a photon counting circuitry with a signal processing arrangement;

FIG. 2 shows an example implementation of an auto-zero comparator;

FIG. 3 shows an example timing diagram for the signal processing arrangement in FIG. 1;

FIG. 4 shows an example implementation of a signal processing arrangement;

FIG. 5 shows an example timing diagram for the signal processing arrangement in FIG. 4;

FIG. 6 shows a further example implementation of a signal processing arrangement;

FIG. 7 shows an example timing diagram for the signal processing arrangement in FIG. 6;

FIG. 8 shows a further example implementation of a signal processing arrangement;

FIG. 9 shows an example timing diagram for the signal processing arrangement in FIG. 8; and

FIG. 10 shows an example implementation of a device for medical diagnostics comprising a photon counting circuitry.

### DETAILED DESCRIPTION

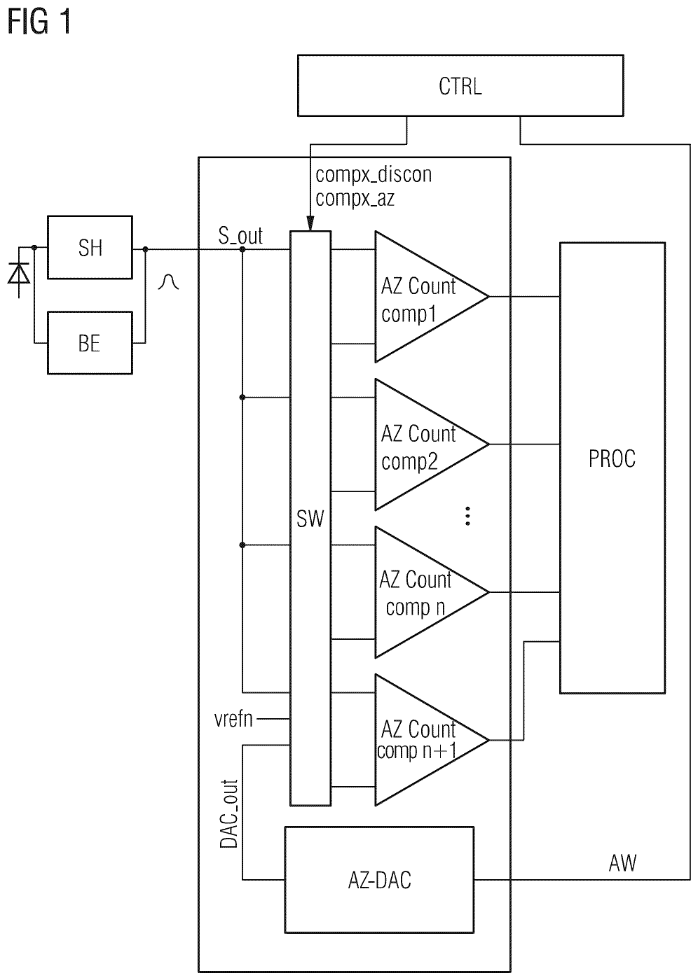

FIG. 1 shows an example implementation of a photon counting circuitry including a signal processing arrangement according to the improved processing concept. Generally, the signal processing arrangement is suitable for an electromagnetic radiation sensor application. The signal processing arrangement comprises a plurality of auto-zero, AZ, comparators comp1, comp2, compn, compn+1, for example such that a number of AZ comparators is in the range from 4 to 10, e.g. in the range from 5 to 8. Each of the AZ comparators has a signal output coupled to a processing block PROC and respective inputs coupled to a switching circuitry SW. A common input S_out of the signal processing arrangement is coupled to the switching circuitry SW, as well as an input for receiving a reference voltage vrefn. The signal processing arrangement further comprises an AZ digital-to-analog converter DAC, AZ-DAC being configured to provide an AZ reference voltage at an AZ reference output based on a predetermined AZ word AW. The AZ reference output DAC_out is also coupled on the input side of the switching circuitry SW.

The processing block is configured to evaluate comparison results provided by the plurality of AZ comparators comp1, comp2, compn, compn+1, e.g. counting comparison events provided by the AZ comparators.

The photon counting circuitry for example comprises a radiation-sensitive element shown in symbolic form as a photodiode in FIG. 1. For example, the radiation-sensitive element provides a current pulse in response to receiving a photon. In other implementations the radiation-sensitive element comprises e.g. a CdZnTe material which absorbs X-rays and directly converts it into an electrical signal.

The output of the radiation-sensitive element is coupled to a signal shaper SH, for example with an optional baseline extraction circuit BE connected anti-parallel to the signal shaper SH. The signal shaper SH generates a voltage pulse from the current pulse received and provides the resulting signal at the common input S_out. The optional baseline extraction circuit BE evaluates the resulting signal at the common input S_out and provides respective feedback to the input of the signal shaper SH.

The implementation of radiation-sensitive elements together with corresponding frontends for providing signals with voltage pulses in photon counting circuitry is well known in the art and will not be explained in more detail herein. Hence, as long as signals with voltage pulses are provided by e.g. a radiation-sensitive element and the corresponding frontend at the common S_out, this signal can be processed with the signal processing arrangement downstream of the common input S_out.

According to the improved processing concept, the signal processing arrangement uses the switching circuitry to “take out” one comparator at a time in a ping-pong scheme to auto-zero its offset and/or low frequency noise, while the rest are operational and performing the comparisons. The switching circuitry SW individually couples the common input S_out and the AZ reference output DAC_out to the signal input of each of the AZ comparators in a switchable fashion. Hence, for example, there is a switch provided for each of the AZ comparators coupling the signal input for a signal to be compared to the common input S_out. Furthermore, there is a switch for each AZ comparator coupling the signal input to the AZ reference output DAC_out.

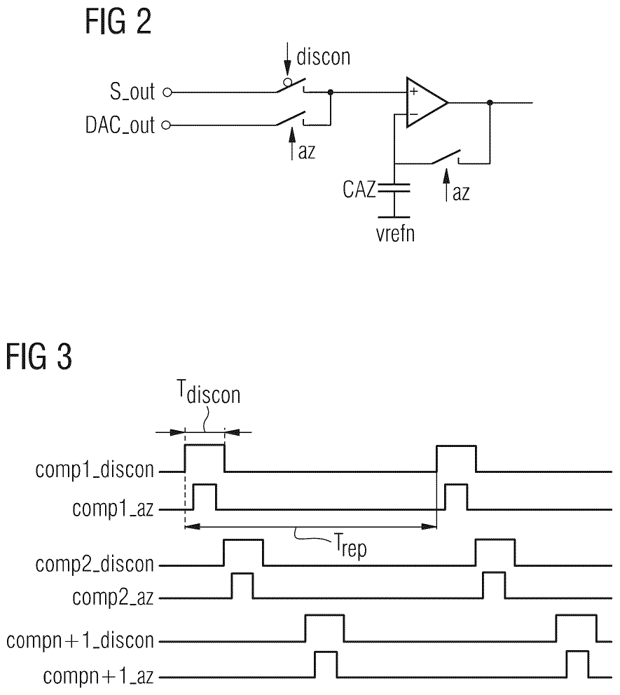

Referring now to FIG. 2, an example implementation of an AZ comparator is shown. The signal input of the comparator is formed by the non-inverting input marked by a plus sign. Both the common input S_out and the AZ reference output DAC_out are coupled to the signal input by respective switches. An AZ capacitor CAZ is connected between a reference input of the comparator, corresponding to the inverting input marked by a minus sign, and a respective reference potential terminal, to which the reference voltage vrefn is provided. The signal output of the comparator is coupled to the reference input via a further switch, thereby establishing a switchable feedback connection.

During normal operation the signal input is connected to the common input S_out, controlled by a respective disconnection signal discon, while the feedback connection and the connection to the AZ reference output DAC_out are opened based on a respective auto-zero signal az. Hence a comparison is performed between the signal at the common input S_out and a threshold voltage of the comparator influenced by a voltage stored on the AZ capacitor CAZ.

In an auto-zero phase, the signal input is disconnected from the common input S_out with a corresponding disconnection signal discon. Furthermore, within the timeframe of when the common input S_out is disconnected, the signal input can be connected to the AZ reference output DAC_out and the feedback connection can be closed based on the auto-zero signal az. Hence, while a respective AZ voltage is provided at the signal input, a corresponding voltage will be stored on the AZ capacitor CAZ due to the feedback connection. This allows any offset and/or low frequency noise, like flicker noise, popcorn noise etc. to be compensated for. Furthermore, drift performance of the comparator can be improved.

Other implementations of an AZ comparator are not excluded by this example. For example, as long as an auto-zero operation based on a respective AZ reference voltage can be performed within a time period when the common input S_out is disconnected from the comparison input, respectively from the signal input of the comparator, other comparator structures could be used as well.

Referring now back to FIG. 1, the control block according to the improved processing concept is configured to disconnect, by controlling the switching circuitry SW, for each of the AZ comparators comp1, comp2, compn, compn+1 in a non-overlapping fashion, the common input S_out from the signal input of the respective AZ comparator during a respective disconnection time, and to connect the AZ reference output DAC_out within the respective disconnection time to the signal input of the respective AZ comparator for a respective AZ time. Hence, at most only one of the comparators is disconnected from the common input S_out at the same time, leaving at least n AZ comparators operational at every time. For example, this is established with respective control signals compx_discon and compx_az, where x is in the range from 1 to n+1, and the extensions discon and az correspond to the signals discon and az, respectively, as described in conjunction with FIG. 2.

It should be noted that the switch connecting the common input S_out to the signal input of the AZ comparator is shown as an opener, such that a high level of the respective control signal opens the switch and achieves the disconnection.

Referring now to FIG. 3, an example switching scheme of a selection of corresponding control signals for the switching circuitry SW is shown. For example, for the first AZ comparator comp1, the corresponding disconnection signal comp1_discon disconnects the signal input of the AZ comparator from the common input S_out during a disconnection time T_discon. Within this disconnection time T_discon the auto-zero signal comp1_az becomes high and controls the connection of the AZ reference output DAC_out to the signal input of the respective AZ comparator for a respective AZ time. The respective AZ time, for example, is smaller than the disconnection time T_discon. Similar control signals are generated for the other comparators, shown for example for comparator 2 with the signals comp2_discon and comp2_az, and for the AZ comparator compn+1 with the signals compn+1_discon, compn+1 _az following the same scheme as described for the first comparator comp1.

As can be seen from the timing diagram of FIG. 3, the disconnection times of the AZ comparators are non-overlapping. Furthermore, the disconnection respective auto-zero operation is repeated with a repetition time T_rep. For example, the repetition time T_rep is chosen to be as long as possible but taking into consideration e.g. a discharging of the AZ capacitor CAZ during the repetition time T_rep.

Since the pulses at the common input S_out can arrive at random times, it can so happen that a potential at the common input S_out is at a different potential than a baseline of an auto-zeroed comparator when connected back. Hence, switching the AZ comparators in and out of the common input S_out can cause glitches.

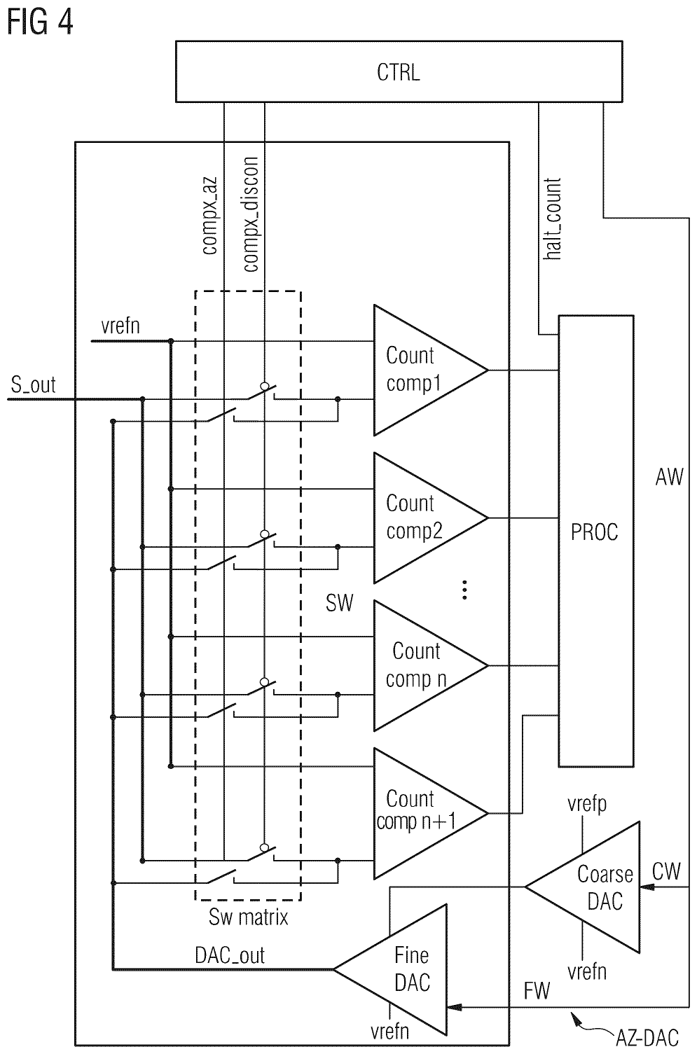

Referring now to FIG. 4, an example implementation of a signal processing arrangement according to the improved concept is shown that is based on the signal processing arrangement implemented in FIG. 1 and considers potential glitching effects. As in FIG. 1, also in the implementation of FIG. 4 the reference voltage vrefn is provided to each of the AZ comparators comp1, comp2, compn, compn+1. Furthermore, the common input S_out and the AZ reference output DAC_out are coupled to the comparator via the switching circuitry SW.

The AZ DAC in this example is implemented with a coarse DAC and a fine DAC that receive different parts of the AZ word AW, namely a coarse word CW and a fine word FW, respectively. Both the coarse DAC and the fine DAC receive the reference voltage vrefn as a negative supply. The coarse DAC receives a supply voltage vrefp as a positive supply and the fine DAC receives the output of the coarse DAC as its positive supply. Other implementations of the AZ DAC are not excluded by this example implementation.

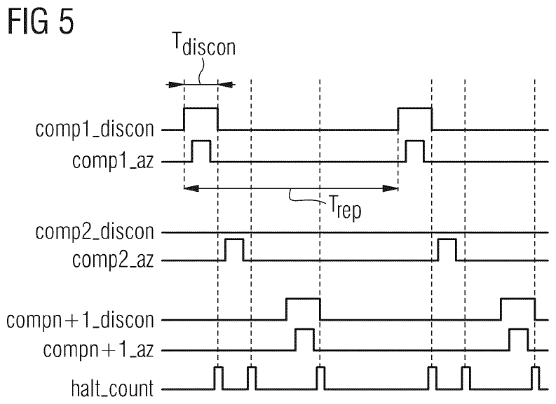

In order to consider potential glitches when switching the AZ comparators in and out, the control block CTRL is further configured to provide a halt indicator halt_count to the processing block PROC at an end of the respective disconnection time T_discon. The halt indicator halt_count may extend over the beginning of the disconnection time of the subsequent comparator being disconnected from the common input S_out. This is, for example, shown in the signal diagram of FIG. 5, which is an extension of the signal diagram of FIG. 3 with the halt indicator halt_count being added. Referring back to FIG. 4, the processing block PROC is configured to halt evaluating of the comparison results, e.g. halt_counting the comparison results, during provision of the halt indicator halt_count, in particular the high signal level of the respective signal.

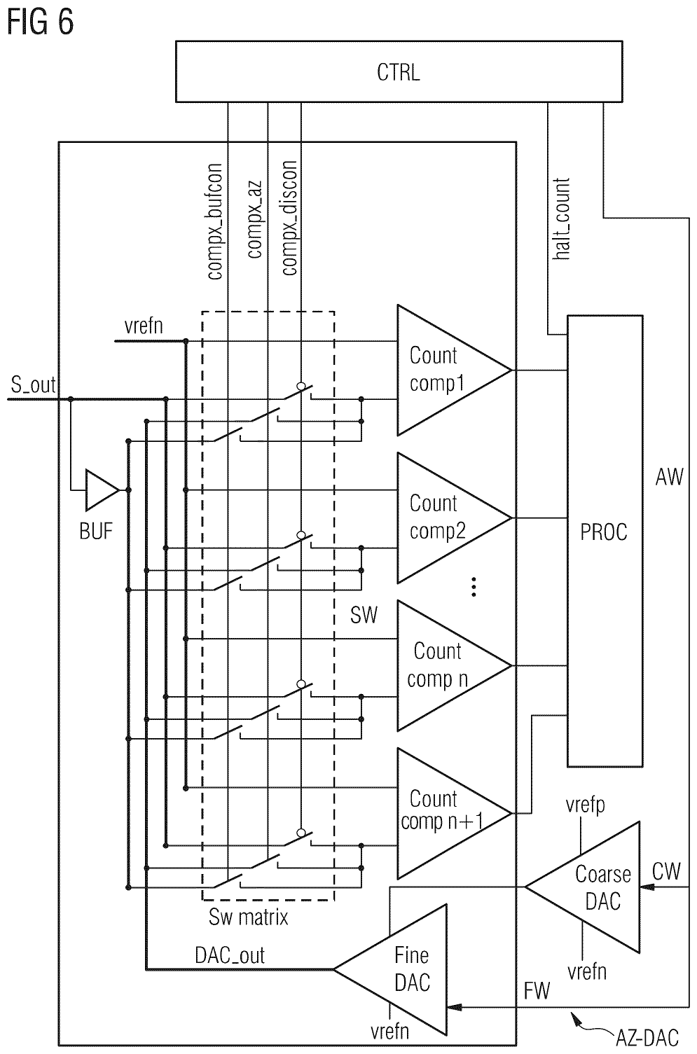

Referring now to FIG. 6, another example implementation of a signal processing arrangement according to the improved processing concept is shown that considers the effect of glitches. The implementation of FIG. 6 is also based on the implementation of the signal processing arrangement as shown in FIG. 1 and further includes the specific implementation of the AZ DAC as described in conjunction with FIG. 4. The switching circuitry SW in the implementation of FIG. 6 further includes respective further switches individually coupling the signal input of each of the AZ comparators, comp1, comp2, compn, compn+1 to the output of a decoupling element BUF which, for example, is implemented as a buffer element. The decoupling element BUF is coupled to the common input S_out at its input side. Hence, any variations at an output of the decoupling element do not directly influence the potential at its input, respectively the common input S_out but are absorbed by the decoupling element BUF. Accordingly, the signal input of each of the AZ comparators is coupled to the common input S_out in a switchable fashion via the decoupling element BUF.

The decoupling element BUF for example is a high speed buffer that has a fast reaction between its input and its output, wherein the term fast is to be seen in relation to rise and fall times of a typical pulse shaped signal at the common input S_out. For example there is no or only insignificant delay between the signals at input and output.

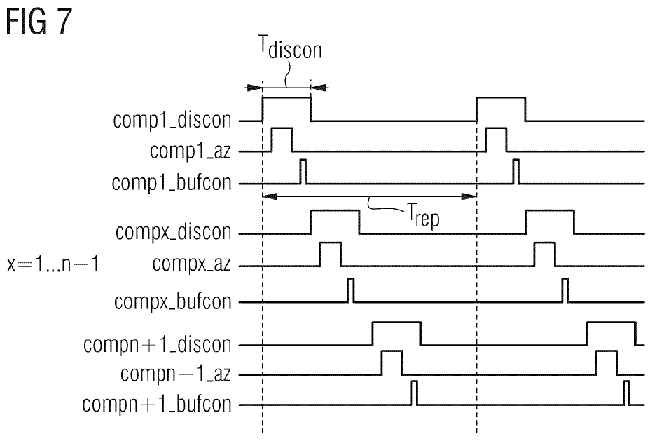

Referring now to FIG. 7, which is based on the signal diagram of FIG. 3, a further control signal with the extension _bufcon is present for each of the comparators. For example, for comparator comp1, the respective control signal comp1 bufcon connects the common input S_out via the decoupling element BUF to the signal input of the AZ comparator comp1 within the respective disconnection time T_discon and after the respective AZ time, e.g., where the control signal comp1_az has a high level. Hence, the potential at the signal input of the comparator comp1 is brought to the level or at least close to the level of the potential at the common input S_out. Hence reconnecting the signal input of the comparator comp1 directly to the common input S_out will not result in a glitch, or at least will reduce the effects of such a glitch.

The same scheme is applied to the other AZ comparators, as can be seen from the rest of the signal diagram of FIG. 7. As in the previous implementations, this procedure can be repeated with the repetition time T_rep.

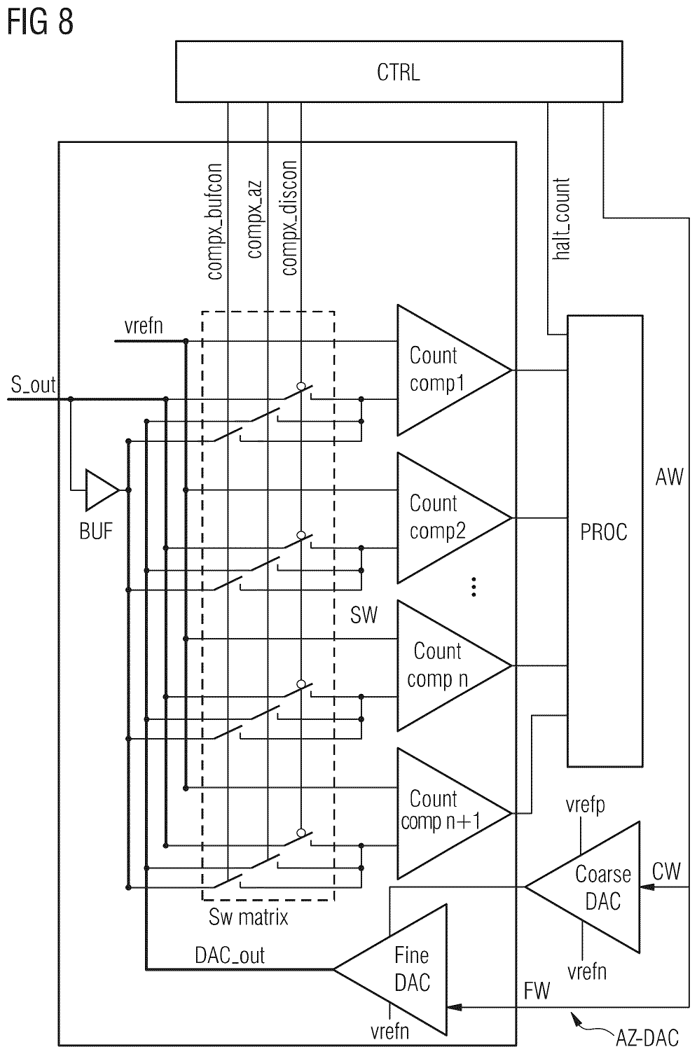

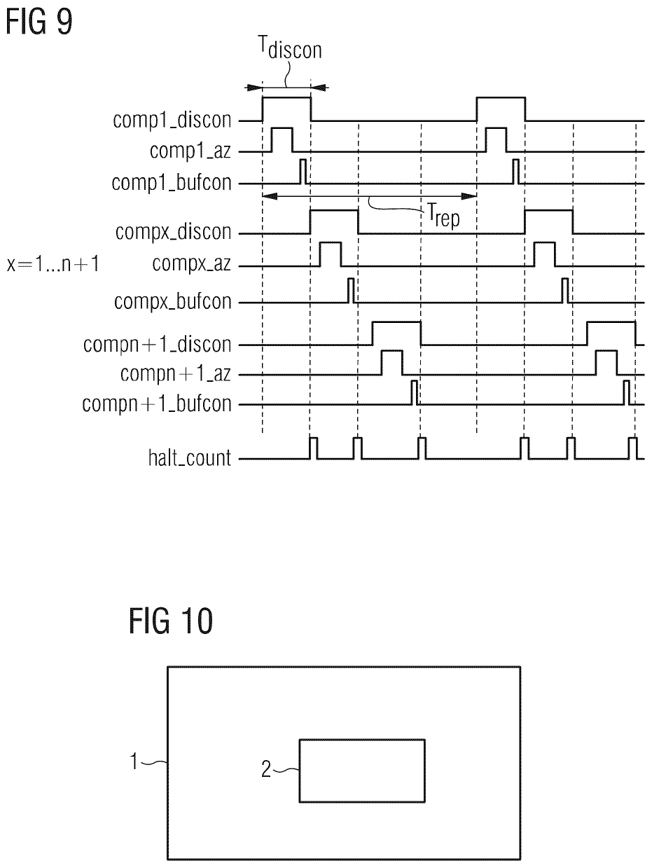

Referring now to FIG. 8, an implementation of a signal processing arrangement according to the improved processing concept is shown, that combines the approaches described in conjunction with FIG. 4 and FIG. 6. Hence, both a halt indicator and a decoupling element are used. A respective switching scheme for control signals provided by the control block CTRL is shown in FIG. 9 and based on the switching scheme shown in FIG. 7 with corresponding halt indicators added.

In the implementation of FIG. 4, where only a halt indicator is used, a duration of the halt indicator, e.g., the high phase of the corresponding signal is in the range between 500 ps and 5000 ps for example, and may even be in the range between 1 ns and 2 ns.

In the implementation of FIG. 8, where potential glitches are reduced by using the decoupling element BUF, the duration of the halt indicator can be reduced compared to the implementation of FIG. 4, e.g. by a factor 10. Hence, the duration of the halt indicator may be in the range between 500 ps and 5000 ps, e.g. in the range between 100 ps and 200 ps.

The AZ word AW may be provided from the control block CTRL. For example, the respective AZ words for each of the comparators may be stored in a memory or may be provided from a specifically programmed FPGA. For example, the AZ words are predetermined during a calibration phase.

FIG. 10 shows an example of an application where a photon counting circuitry 2 equipped with the signal processing arrangement according to one of the implementations described above is provided in a device 1 for medical diagnostics. The device 1 for medical diagnostics may be configured, for example, as an X-ray apparatus or a computed tomography scanner.

The embodiments of the improved processing concept disclosed herein have been discussed for the purpose of familiarizing the reader with novel aspects of the implementation of the improved processing concept. Although some embodiments have been shown and described, many changes, modifications, equivalents and substitutions of the disclosed concepts may be made by one having skill in the art without unnecessarily departing from the scope of the claims.

In particular, the implementation of the improved processing concept is not limited to the disclosed embodiments, and gives examples of many alternatives as possible for the features included in the embodiments discussed. However, it is intended that any modifications, equivalents and substitutions of the disclosed concepts be included within the scope of the claims which are appended hereto.

Features recited in separate dependent claims may be advantageously combined. Moreover, reference signs used in the claims are not limited to be construed as limiting the scope of the claims.

Furthermore, as used herein, the term “comprising” does not exclude other elements. In addition, as used herein, the article “a” is intended to include one or more than one component or element, and is not limited to be construed as meaning only one.

What is claimed is:

1. A signal processing arrangement for an electromagnetic radiation sensor application, the signal processing arrangement comprising
a common input;
a plurality of auto-zero, AZ, comparators, wherein each of the AZ comparators comprises
a reference potential terminal for receiving a reference voltage;
a signal input for receiving a voltage to be compared; and
a signal output coupled to a processing block;
the processing block configured to evaluate comparison results provided by the plurality of AZ comparators;
an AZ digital-analog-converter, DAC, configured to provide an AZ reference voltage at an AZ reference output based on a predetermined AZ word;
switching circuitry configured to individually couple the common input and the AZ reference output to the signal input of each of the AZ comparators in a switchable fashion; and
a control block configured to, by controlling the switching circuitry,
disconnect, for each of the AZ comparators, the common input from the signal input of the respective AZ comparator during a respective disconnection time; and
connect the AZ reference output within the respective disconnection time to the signal input of the respective AZ comparator for a respective AZ time.

2. The signal processing arrangement according to claim 1, wherein the control block is configured to perform the disconnecting of each of the AZ comparators sequentially.

3. The signal processing arrangement according to claim 2, wherein the control block is configured to perform the sequential disconnecting repeatedly with a predetermined repetition time.

4. The signal processing arrangement according to claim 1, wherein
the control block is further configured to provide a halt indicator to the processing block at an end of the respective disconnection time; and
the processing block is configured to halt evaluating of the comparison results during provision of the halt indicator.

5. The signal processing arrangement according to claim 4, wherein a duration of the halt indicator is in the range between 500 ps and 5000 ps, in particular in the range between 1 ns and 2 ns.

6. The signal processing arrangement according to claim 1, wherein
the switching circuitry is further configured to individually couple the common input to the signal input of each of the AZ comparators in a switchable fashion via a decoupling element; and
the control block is further configured to, by controlling the switching circuitry, connect the common input via the decoupling element to the signal input of the respective AZ comparator within the respective disconnection time and after the respective AZ time.

7. The signal processing arrangement according to claim 4, wherein,
the switching circuitry further configured to individually couples the common input to the signal input of each of the AZ comparators in a switchable fashion via a decoupling element;
the control block is further configured to, by controlling the switching circuitry, connect the common input via the decoupling element to the signal input of the respective AZ comparator within the respective disconnection time and after the respective AZ time; and
a duration of the halt indicator is in the range between 50 ps and 500 ps.

8. The signal processing arrangement according to claim 1, wherein each AZ comparator comprises
an AZ capacitor connected between a reference input of the respective AZ comparator and the respective reference potential terminal; and
a switchable feedback connection between the respective reference input and the respective signal output, wherein the control block is configured to enable the respective feedback connection during the respective AZ time.

9. The signal processing arrangement according to claim 1, wherein the AZ word is different for each AZ comparator.

10. The signal processing arrangement according to claim 1, wherein the processing block is configured to count events based on the comparison results provided by the plurality of AZ comparators.

11. The signal processing arrangement according to claim 1, wherein a number of the AZ comparators is in the range from 4 to 10.

12. A photon counting circuitry, comprising:
a signal processing arrangement according to claim 1,
a photon detector being configured to generate a current pulse upon reception of a photon; and
a signal shaper coupled to the photon detector and configured to generate a voltage pulse at the common input based on a received current pulse.

13. A device for medical diagnostics, comprising:
at least one photon counting circuitry according to claim 12,
wherein the device is configured as an X-ray apparatus or a computed tomography scanner.

14. A signal processing method for an electromagnetic radiation sensor application with a common input and a plurality of auto-zero, AZ, comparators, wherein each of the AZ comparators comprises:
a signal input for receiving a voltage to be compared, the signal input being switchably coupled to the common input; and
a signal output for providing a comparison result;
the method comprising:
disconnecting, for each of the AZ comparators, the common input from the signal input of the respective AZ comparator during a respective disconnection time;
providing, within the respective disconnection time, an AZ reference voltage based on a respective predetermined AZ word to the signal input of the respective AZ comparator for a respective AZ time; and
evaluating of the comparison results provided by the plurality of AZ comparators.

15. The method according to claim 14, further comprising
providing a halt indicator at an end of the respective disconnection times; and
halting the evaluating of the comparison results during provision of the halt indicator.

16. The method according to claim 14, further comprising connecting the common input via a decoupling element to the signal input of the respective AZ comparator within the respective disconnection time and after the respective AZ time.
